(19) United States
(12) Patent Application Publication
(10) Pub. No.: US 2008/0206844 A1
(43) Pub. Date: Aug. 28, 2008

(54) Method and Apparatus for Treatment of Gases

(76) Inventors: Thomasz Rudas, Bentley (AU); Martin Richard Gravett, Bentley (AU)

Correspondence Address: DUANE MORRIS, LLP;IP DEPARTMENT, 30 SOUTH 17TH STREET, PHILADELPHIA, PA, 19103-4196, US

(21) Appl. No.: 11/911,791

(22) PCT Filed: Apr. 18, 2006
(86) PCT No.: PCT/AU2006/000510

§ 371 Date: Mar. 27, 2008

(30) Foreign Application Priority Data
Apr. 18, 2005 (AU) 2005901924

(51) Int. Cl. C12S 5/00; C12M 1/04; C12M 1/34

(52) U.S. Cl. 435/266; 435/283.1; 435/287.1

(57) ABSTRACT

A method and apparatus for the treatment of contaminated gas which comprises placing a biofilter material (28) into a vessel (10) which is arranged to be sealed. A compressor (14) and valve (16) are provided for feeding pressurised contaminated gas into the vessel (10). A valve (20) is provided for releasing treated gas from the vessel (10). The use of pressurised contaminated gas increases the pressure in the vessel (10) which ensures a substantially even distribution of gas in the vessel (10) and the biofilter material (28). The treatment reduces the level of contaminants in the gas.

### FIELD OF THE INVENTION

The present invention relates to a method and apparatus for treatment of gases.

### SUMMARY OF THE INVENTION

In accordance with one aspect of the present invention there is provided a method for treatment of gases characterised by placing a biofilter substrate material in a vessel which is arranged to be sealed, feeding pressurised contaminated and/or odorous gas into the vessel whilst it is sealed so as to increase the pressure inside the vessel above atmospheric pressure, and continuing the feeding of the pressurised gas until the inlet pressure and the internal pressure in the vessel are substantially equalised thereby ensuring a substantially even distribution of gas in the vessel and biofilter substrate material, such that, concentration of malodorous material or undesirable compounds in the gas is diminished biologically.

In accordance with a further aspect of the present invention there is provided an apparatus for the treatment of gases characterised by a vessel which is able to be sealed, the vessel being arranged to contain a biofilter substrate material, means being provided for feeding pressurised gas into the vessel, and means for releasing gas from the vessel.

### BRIEF DESCRIPTION OF THE DRAWINGS

The present invention will now be described, by way of example, with reference to the accompanying drawing which is a schematic side elevation of a pressure vessel of the present invention

### DETAILED DESCRIPTION OF THE INVENTION

Biofiltration is a technique for biological elimination of malodorous gas emissions and of low concentrations of volatile organic compounds (VOCs). A biofilter achieves this function by bringing pollutants contained in a gaseous effluent stream into contact with microorganisms that have an ability to degrade such pollutants. A biofilter vessel may contain a substrate material in the form of a filter material, porous media, packing or “bed” material that provides a moisture rich surface upon which these microorganisms live. The substrate material may contain activated carbon, plastic, wood chips, sawdust, compost, glass fibre, rock wool, perlite, shredded tree waste, or any combination of these or similar materials. A biofilter may operate under aerobic, anoxic or anaerobic conditions, or a combination of these, depending on the pollutant targeted for removal.

A microbiological population inside a vessel that is performing an aerobic biofiltration function requires oxygen to maintain metabolic activity. The constant supply of this oxygen is crucial in maintaining optimum microbial activity, thus ensuring rapid biodegradation of particulates, various chemical compounds and organic matter (eg. hazardous and/or odorous compounds in process air). In a biofilter, oxygen is typically delivered through input of contaminated and/or odorous air. The difficulty in delivering and distributing contaminated and/or odorous input air evenly using conventional biofilter systems with organic and/or inorganic media is as follows:

1. In-vessel—Forced Aeration:
Large volumes of air are blown (or sucked) into a material mass at low pressure. This method relies on the biofilter media material maintaining sufficient porosity to allow air to penetrate the entire volume of material without significant pressure drop. The disadvantages include:
   Air will usually find the path of least resistance, thereby aerating some sections of the material, but leaving other sections with insufficient oxygen, especially with materials that have a high bulk density, become saturated with moisture, or are filled to increased depth (i.e. low permeability).
   Large volume of incoming air cools the material near areas of high flow resulting in an uneven temperature distribution in the biofilter media or bed.
   Large volumes of air remove moisture from the material, which may be undesirable.
   Blockage of any of the air delivery points results in that section of material not receiving sufficient oxygen and reduced overall biofiltration capacity and system efficiency.
   Development of preferential flow paths or “channels” result in less than optimal contact or residence times, and therefore reduced efficiency.
   Special proprietary “engineered” media material may be required, which can be expensive.

2. Open bed—Forced Aeration:
Biofilter media material is piled in channels or bays with ducting and/or grating beneath the pile or bed. Biofilter beds are generally covered (under roof) to reduce moisture loss from evaporation. The disadvantages of this system of aeration include:
   Pile size (height) is restricted because increasing pile size reduces the effectiveness of aeration, as material deep inside the pile does not receive a fresh oxygen supply.
   Material blend is also restricted, as high bulk density materials don't have sufficient porosity to allow adequate airflow. Therefore this method is typically only suited to low bulk density materials such as shredded tree waste, woodchips or proprietary “engineered” media materials.
   Requirement for maintaining permeability limits biofilter media fill or bed depth and therefore requires large land area to treat large volumes of contaminated and/or odorous air.
   Development of preferential flow paths or “channels” result in less than optimal contact or residence times.
   Maintaining the temperature in the optimal range within the pile is difficult.
   Management of moisture in the pile is complicated by the effect of ambient temperature, humidity and wind.

The present invention will now be described with particular reference to an embodiment involving treatment of an odour contaminated aerobic gas stream.

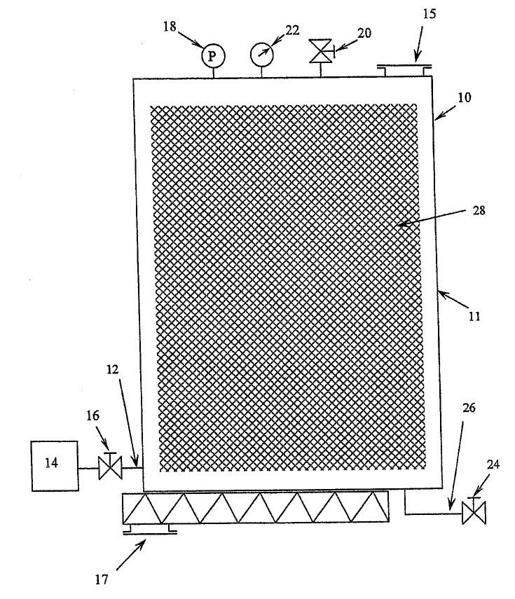

The pressure vessel 10 shown in FIG. 1 comprises an outer body 11 which is arranged to be sealed off. The body 11 is provided with an air supply pipe 12 about which is mounted a compressor 14. The air supply pipe 12 is provided with an inlet valve 16.

The vessel 10 may be provided with a plurality of air supply pipes 12 together with respective compressors 14 and inlet valves 16.

Pressure regulation means 18 is provided at an upper end of the body 11. Further, adjacent to the pressure regulation means 18 there is provided an automated and controlled exhaust valve 20 and measurement means 22 for measuring the concentration of the various gasses in the vessel 10 such as oxygen, carbon dioxide or other gases.

Further, the vessel 10 is arranged to be sealed in use. The sealing may be effected by conventional means such as by employing flanges and bolted openings with a conventional gasket.

Thus, unless either valve 16 or 20 is open the vessel 10 is sealed during normal operation.

Further, adjacent a lower end of the body 11 there is provided a drain valve 24 mounted in a pipe 26 for removal of liquid such as condensate. It is possible to monitor the acidity or alkalinity of the contents of the vessel by measuring the pH of the removed liquid.

In operation, the vessel 10 is loaded in known manner with organic and/or inorganic biofilter substrate material 28 by an inlet means 15 and an outlet means 17. For example, the vessel 10 may be provided with an auger or other mechanical conveying arrangement. Alternatively, an end of the vessel may be arranged to be opened such as by a flanged gate or sealing door, wherein the used biofilter substrate material 28 can be removed and fresh material loaded into vessel 10. The biofilter substrate material contains aerobic microorganisms suitable for treatment of gases in the oxygen containing atmosphere.

Contaminated and/or odorous air is introduced into the vessel 10 through the air supply pipe 12 and the valve 16. By means of the compressor 14 the pressure of the incoming air is increased to above atmospheric pressure such as 1 to 1000 kPa above atmospheric pressure, preferably 1 to 100 kPa, more preferably 10 to 50 kPa above atmospheric pressure.

The oxygen and/or carbon dioxide content of the vessel 10 is monitored by the measurement means 22. When a certain predetermined condition is reached the exhaust valve 20 is automatically opened so as to release air from the vessel 10 and reduce the pressure inside the vessel 10 typically to atmospheric pressure. A typical predetermined condition which is reached could be oxygen content and/or carbon dioxide content, or a predetermined contact or residence time.

Subsequently, following reduction of pressure in the vessel 10 additional pressurised contaminated and/or odorous air is again introduced automatically through the pipe 12 by the valve 16 and the pressure inside the vessel 10 is again raised above atmospheric pressure to the range from 1 to 1000 kPa. It is possible to achieve continuous treatment of contaminated and/or odorous air through connecting a plurality of the vessels 10 in parallel and/or series.

The valves 12 and 16 are gas tight and are arranged to be automatically opened and closed in sequence. In this connection the valves 12 and 16 are typically arranged to be controlled by an automated process control means.

The biofilter substrate material in the vessel 10 may be removed periodically through the outlet 17 as described above and replaced as required for maintaining satisfactory performance. If desired, the pH of the contents of the vessel may be adjusted by addition of a suitable acid or alkaline solution. Any condensate or other liquid may be removed through the valve 24 and the pipe 26.

In the operation of the present invention, gas is not blown into the vessel 10 at low pressure so as to promote flow only, which may result in the gas finding the path of least resistance. In the present invention, pressure in the vessel 10 is equalised. The air supply pipe 12 is pressurised whilst initially the pressure in the vessel 10 is atmospheric. By forcing pressurised air into the vessel 10 the pressure therein becomes equalised throughout at a pressure above atmospheric pressure. The entire volume of the vessel 10 can be considered to have received the contaminated and/or odorous air when the pressure in the pipe 12 has equalised with the pressure in the vessel 10. In this condition, every free space inside the vessel 10 contains the contaminated and/or odorous air. Thus, the air eventually reaches all points within the biofilter material. The time required for this depends on the size of vessel 10, as well as the volume, permeability and bulk density of the biofilter substrate material.

Further, the time required for equalisation may be varied by increasing pressure inside the pipe 12. This leads to a greater initial pressure difference between the pipe 12 and the vessel 10, thus increasing air flow and leading to a reduction in equalisation time.

In accordance with the present invention the contaminated and/or odorous air is evenly distributed throughout the vessel 10. This causes an even temperature distribution throughout the biofilter material as substantially all the biofilter material is exposed to the contaminated and/or odorous air. Also, the efficiency of the treatment is enhanced because the contaminated and/or odorous air is spread substantially evenly throughout the entire vessel 10.

Further, addition of further contaminated and/or odorous air is achieved efficiently which reduces the volume of biofilter substrate material required. Also, permissible bed depth of the biofilter substrate material is increased reducing the amount of space required compared to prior art systems.

Still further, homogeneous conditions are created for the microbial population in the vessel 10 without the need for additional mechanical agitation or moisture addition.

Selected microbial inoculants (fungi and/or bacteria or other specifically cultured microorganisms) can be introduced to the biofilter media material to specifically degrade target compounds (e.g. sulfur oxidising or sulfate reducing bacteria).

The apparatus and method described above relate to aerobic treatment of contaminated and/or odorous air. However, the present invention is equally applicable to anoxic and anaerobic treatment or even alternating aerobic, anoxic and anaerobic treatments. Thus, the present invention is applicable to the treatment of gaseous effluent streams or the like not containing air such as biogas, synthesis gas and chemical vapours.

Modifications and variations such as would be apparent to a skilled addressee are deemed to be within the scope of the present invention.

1. A method for treatment of gases characterised by placing a biofilter substrate material in a vessel which is arranged to be sealed, feeding pressurised contaminated and/or odorous gas into the vessel whilst it is sealed so as to increase the pressure inside the vessel above atmospheric pressure, and continuing the feeding of the pressurised gas until the inlet pressure and the internal pressure in the vessel are substantially equalised thereby ensuring a substantially even distribution of gas in the vessel and biofilter substrate material such that, the concentration of malodorous material or undesirable compounds in the gas is diminished biologically.

2. A method according to claim 1, characterised in that after a predetermined condition is reached gas is released from the vessel to reduce the pressure therein.

3. A method according to claim 2, characterised in that the predetermined condition is a gas concentration or contact or residence time.

4. A method according to claim 2, characterised in that the vessel is subsequently resealed and more contaminated and/or odorous air is fed into the vessel for treatment.

5. A method according to claim 1, characterised in that the internal pressure in the vessel during treatment is 1 to 1000 kPa above atmospheric pressure.

6. A method according to claim 5, characterised in that the internal pressure in the vessel during treatment is 1 to 100 kPa above atmospheric pressure.

7. A method according to claim 1, characterised in that the pressure inside the vessel is monitored and/or controlled.

8. A method according to claim 1, characterised in that the treatment is aerobic and the contaminated and/or odorous gas contains oxygen.

9. A method according to claim 8, characterised in that the gas is air.

10. A method according to claim 1, characterised in that the treatment is anoxic or anaerobic.

11. A method according to claim 1, characterised in that the distribution of gas in the vessel is achieved without mechanical agitation.

12. An apparatus for the treatment of gases characterised by a vessel which is able to be sealed, the vessel being arranged to contain a biofilter substrate material, means being provided for feeding pressurised gas into the vessel, and means for releasing gas from the vessel.

13. An apparatus according to claim 12, characterised in that means is provided for monitoring and/or controlling the pressure inside the vessel.

14. An apparatus according to claim 12, characterised in that means is provided for draining liquid from inside the vessel.

15. An apparatus according to claim 12, characterised in that means is provided for assessing vessel internally for insertion and removal of biofilter substrate material.

16. An apparatus according to claim 12, characterised in that means is provided for measuring the concentration of gases in the vessel.

17. An apparatus according to claim 12, characterised in that means is provided for measuring concentration of various gases or measuring the contact or residence time.
